(19) United States
(12) Patent Application Publication
(10) Pub. No.: US 2022/0023271 A1
(43) Pub. Date: Jan. 27, 2022

(54) COMPOSITION FOR PREVENTING OR TREATING CELLULAR SENESCENCE-RELATED DISEASES COMPRISING ZOTAROLIMUS AS ACTIVE INGREDIENT

(71) Applicant: RESEARCH COOPERATION FOUNDATION OF YEUNGNAM UNIVERSITY, Gyeongsan-si (KR)

(72) Inventors: Jae-Ryong KIM, Daegu (KR); Eok-Cheon KIM, Wonju-si (KR); Kyong-Jin JUNG, Daegu (KR); You Lim SON, Daegu (KR)

(73) Assignee: RESEARCH COOPERATION FOUNDATION OF YEUNGNAM UNIVERSITY, Gyeongsan-si (KR)

(21) Appl. No.: 17/413,064

(22) PCT Filed: Oct. 11, 2019
(86) PCT No.: PCT/KR2019/013372

§ 371 Date: Jun. 11, 2021

(30) Foreign Application Priority Data
Dec. 13, 2018 (KR) 10-2018-0161043

(51) Int. Cl. A61K 31/436; A61P 9/10; A61P 11/00

(52) U.S. Cl. CPC A61K 31/436; A61P 11/00; A61P 9/10

(57) ABSTRACT

The present invention relates to a composition for preventing or treating cellular senescence-associated diseases comprising zotarolimus as an active ingredient, and in more detail, it was confirmed that zotarolimus exhibits a senomorphics effect of restoring the function and morphology of fibroblasts, umbilical vascular endothelial cells, renal tubular cells and retinal pigmented epithelial cells, in which senescence is induced and exhibits an effect of improving tissue fibrosis induced by cellular aging through the senomorphics effect and thus the composition comprising zotarolimus as an active ingredient can be provided as a composition for treating cellular senescence-associated diseases.

### CROSS REFERENCE TO RELATED APPLICATIONS

This application is a U.S. National Stage Application of International Application No. PCT/KR2019/013372, filed on Oct. 11, 2019, which claims the benefit under 35 USC 119(a) and 365(b) of Korean Patent Application No. 10-2018-0161043, filed on Dec. 13, 2018, in the Korean Intellectual Property Office, the entire disclosure of which is incorporated herein by reference for all purposes.

### TECHNICAL FIELD

The present invention relates to a composition for preventing or treating cellular senescence-associated diseases comprising as an active ingredient zotarolimus, which exhibits an effect of restoring senescent cells to normal cells.

### BACKGROUND ART

Senescent cells accumulate in individual tissues and organs with aging, and accumulation of senescent cells not only induces changes in the function and structure of tissues and organs due to aging, but also plays an important role in the etiology of various senescence-associated diseases such cancer, diabetes, obesity, tissue fibrosis, senile eye diseases, cardio-cerebrovascular disease, degenerative brain disease, osteoarthritis, skin aging and chronic skin wounds, etc. Therefore, it has been suggested that delaying or overcoming aging is the most effective method for the prevention and treatment of senescence-associated diseases such as cancer, diabetes, and cardiovascular disease.

Rapamycin, SIRT1 activator, calorie-limiting mimetic, AMPK activator, and telomerase activator are promising as aging control drugs. In addition, recently, senotherapeutics, which target senescent cells, have been developed and their efficacy has been reported at the cellular level and in animal models. Senotherapeutics are divided into senolytics, which selectively kill only senescent cells, and senomorphics, which restore the function or shape of senescent cells like young cells.

Quercetin and dasatinib, which are Bcr-Abl protein kinase inhibitors, ABT263 and ABT737, which are Bcl-2 kinase inhibitors, and A1331852 and A1155463, which are BCL-XL inhibitors, and UBX0101, which is an MDM2/p53 inhibitor, FOXO4-DRI, which is p53 inhibitor, and 17-DMAG, which is HSP90 inhibitor, have been recently reported as senolytics. As senomorphics, mTOR inhibitors such as rapamycin, IKK/NFkB inhibitors, free radical scavengers and JAK inhibitors have been reported.

Accordingly, research and development for preventing or treating senescence-associated diseases are actively progressing through the development of senotherapeutics that target senescence and senescent cells.

Zotarolimus is a drug used as a treatment for restenosis of the coronary artery and it is reported to inhibit cell growth by binding to mTOR in the cell and inhibiting its activity, however until now, there has been no report on the effect of senomorphics, which targets senescent cells and restores the function or morphology of senescence-induced cells.

### DISCLOSURE

### Technical Problem

The present invention provides a composition comprising as an active ingredient to restore the function and morphology of senescent cells to provide a composition for improving or treating diseases caused by cellular aging.

### Technical Solution

The present invention provides a senomorphics composition comprising zotarolimus as an active ingredient.

The present invention provides a pharmaceutical composition for preventing or treating cellular senescence-associated diseases comprising zotarolimus as an active ingredient.

In addition, the present invention provides a composition for improving aging or extending life comprising zotarolimus as an active ingredient.

### Advantageous Effects

According to the present invention, it was confirmed that a composition comprising zotarolimus as an active ingredient exhibits a senomorphics effect of restoring the function and morphology of fibroblasts, umbilical vascular endothelial cells, renal tubular cells and retinal pigmented epithelial cells, in which senescence is induced and exhibits an effect of improving tissue fibrosis induced by cellular aging through the senomorphics effect and thus the composition comprising zotarolimus as an active ingredient can be provided as a composition for treating cellular senescence-associated diseases.

### DESCRIPTION OF DRAWINGS

FIGS. 1A to 1C show results of confirming the senomorphics action of zotarolimus in premature senescent fibroblasts by doxorubicin; FIG. 1A shows a result of treatment with 100 nM rapamycin, 100 nM ABT263 and 100 nM zotarolimus on young fibroblasts (HDF) and premature senescent fibroblasts by doxorubicin, and SAβG activity staining after 4 days; FIG. 1B shows a result of the SAβG activity staining level; and FIG. 1C shows a result of confirming cell growth. HDF=human dermal fibroblasts, Young=young cells, Dox=doxorubicin, NT=0.01% DMSO, Rapa=100 nM rapamycin, ABT=100 nM ABT263, Zota=100 nM zotarolimus (** p<0.01, * p<0.05).

FIGS. 2A to 2D show results of confirming the senomorphics action of zotarolimus in replicative senescent fibroblasts; FIG. 2A and FIG. 2B show a result of treatment with 100 nM rapamycin and 100 nM zotarolimus on young fibroblast (HDF) and replicative senescent fibroblasts, and SAβG activity staining after 4 days; FIG. 2C shows a result of confirming the cell growth level according to the concentration of zotarolimus; FIG. 2D shows a result of confirming the activity of LDH in the cell culture solution. HDF=human dermal fibroblasts, Young=young cells, Senescent=replicative senescent cells, NT=0.01% DMSO, Rapa=100 nM rapamycin, ABT=100 nM ABT263, Zota=100 nM zotarolimus (*, p<0.05; **, p<0.01).

FIGS. 3A to 3C show results of confirming the senomorphics action of zotarolimus in premature senescent vascular endothelial cells by doxorubicin; FIG. 3A shows a staining picture of treatment with a 100 nM rapamycin, 100 nM ABT263 and 100 nM zotarolimus on young vascular endothelial cells (HUVEC) and premature senescent vascular endothelial cells by doxorubicin, and performing SAβG activity staining after 4 days; FIG. 3B shows a result of the level of SAβG activity staining; and FIG. 3C shows a result of confirming cell growth level. HUVEC=human umbilical vascular endothelial cells, Young=young cells, Dox=doxorubicin, NT=0.01% DMSO, Rapa=100 nM rapamycin, ABT=100 nM ABT263, Zota=100 nM zotarolimus (*, p<0.05; **, p<0.001).

FIGS. 4A to 4D show results of confirming the senomorphics action of zotarolimus in replicative senescent vascular endothelial cells; FIG. 4A shows a result of confirming the treatment with 100 nM rapamycin, 100 nM ABT263 and 100 nM zotarolimus on replicative senescent vascular endothelial cells (HUVEC), and performing SAβG activity staining after 4 days; FIG. 4B shows a result of the SAβG activity staining level; FIG. 4C shows a result of confirming the cell growth level according to the concentration of zotarolimus; and FIG. 4D shows a result of confirming the activity of LDH in the cell culture solution. HUVEC=human umbilical vascular endothelial cells, Young=young cells, Senescent=replication senescent cells, NT=0.01% DMSO, Rapa=100 nM rapamycin, ABT=100 nM ABT263, Zota=100 nM zotarolimus (*, p<0.05; **, p<0.01).

FIGS. 5A to 5D show results of confirming the senomorphics action of zotarolimus in premature senescent retinal pigmented epithelial cells by doxorubicin; FIG. 5A shows a result of treatment with 100 nM rapamycin and 100 nM zotarolimus in premature senescent retinal pigmented epithelial cells by doxorubicin, and performing SAβG activity staining after 4 days; FIG. 5B shows a result of the level of SAβG activity staining; FIG. 5C shows a result of confirming the cell growth level in a concentration-dependent manner of zotarolimus; and FIG. 5D shows a result of confirming the cytotoxic effect through LDH activity analysis. ARPE=adult retinal pigmented epithelial cells, Young=young cells, Dox=doxorubicin, Senescent=premature senescent cells by doxorubicin, NT=0.01% DMSO, Rapa=100 nM rapamycin, ABT=100 nM ABT263, Zota=100 nM zotarolimus (**, p<0.01).

FIGS. 6A to 6C show results of confirming the senomorphics action of zotarolimus in premature senescent renal tubular cells by doxorubicin; FIG. 6A shows a result of treatment with 100 nM ABT263, 100 nM rapamycin, 100 nM zotarolimus on premature senescent human renal tubular cells (HK2) by doxorubicin, and confirming the cell growth level after 4 days; FIG. 6B shows a result of CCK-8 analysis confirming the cell growth level. HK2=human tubular epithelial cells, Dox=doxorubicin, NT=0.01% DMSO, Rapa=100 nM rapamycin, ABT=100 nM ABT263, Zota=100 nM zotarolimus (**, p<0.01); FIG. 6C shows that SAβG staining decreased.

FIGS. 7A to 7M show results of confirming the efficacy of zotarolimus on renal fibrosis induced by renal ischemia-reperfusion injury in mice; FIG. 7A is a schematic diagram showing the experimental process; FIG. 7B shows a result of confirming the weight change before the experiment and after kidney resection; FIG. 7C shows a result of plasma creatinine concentration; FIG. 7D shows a result of the plasma BUN concentration; FIG. 7E shows a result of hematoxylin-eosin staining, trichrome staining and PAS staining of the tissue sample; FIG. 7F shows a result of the degree of fibrosis of the tissue after trichrome staining in the sample; FIG. 7G shows a result of confirming the degree of cellular aging in the tissue by Sudan Black B staining; FIG. 7H shows a result of confirming the lipid peroxide level in the tissue; FIG. 7I shows a result of performing MDA analysis in the tissue; FIG. 7J shows a result of confirming the level of superoxides in the tissue; FIG. 7K shows a result of confirming the expression level of 4-HNE and p16 protein in the tissue by Western blot analysis; FIG. 7L shows a result of Western blot analysis of tissue proteins, and FIG. 7M shows a result of densitometry analysis of western blot result. Sham=no ischemia-reperfusion injury, PBS=ischemia-reperfusion injury, Zota=ischemia-reperfusion injury followed by zotarolimus intraperitoneal injection, UIRI=left kidney ischemia-reperfusion injury, UNx=right kidney resection, PAS=periodic acid-Schiff, MDA=malondialdehyde, 4-HNE=4-hydroxynonenal, pRb=phosphorylated Rb, COL1=collagen type I, α-SMA=alpha-smooth muscle actin, SOD2=superoxide dismutase 2 (**, p<0.01).

FIGS. 8A to 8D show results of confirming the effect of zotarolimus in a mouse animal model in which lung fibrosis is induced by bleomycin in mice; FIG. 8A is a schematic diagram showing an experimental process; FIG. 8B shows a result of confirming a change in body weight in the experimental process; FIG. 8C shows a result of performing Hematoxylin-eosin and trichrome staining in a lung tissue sample; and FIG. 8D shows a result of confirming the degree of lung fibrosis in a tissue sample. D=zotarolimus or PBS intraperitoneal injection, NT=not treated, PBS=PBS intraperitoneal injection, Zota=zotarolimus intraperitoneal injection (**, p<0.01).

FIGS. 9A to 9D show results of confirming the efficacy of zotarolimus on peritoneal fibrosis caused by CHG in mice; FIG. 9A is a schematic diagram showing an experimental process; FIG. 9B shows a result of confirming a change in body weight in the experimental process; FIG. 9C shows a result of performing Hematoxylin-eosin and trichrome staining in an abdominal wall tissue sample; and FIG. 9D shows a result of confirming the thickness of peritoneal mesothelial cell layer in a tissue sample. CHG=chlorhexidine gluconate, D=zotarolimus or DMSO intraperitoneal injection, NT=not treated, DMSO=DMSO intraperitoneal injection, Zota=zotarolimus intraperitoneal injection (*, p<0.05; **, p<0.01).

### BEST MODE

Hereinafter, the present invention will be described in more detail.

As it is reported that when senescence cells present in tissues are removed from animal models, the structure and function of tissues and organs due to aging are improved to treat senescence-associated diseases, the health life is increased, the inventors of the present invention conducted research on senotherapeutics targeting senescent cells to confirm that zotarolimus restores the function or morphology of senescent cells to normal cells, thereby improving diseases caused by cellular aging and completed the present invention.

The present invention can provide a senomorphics composition comprising zotarolimus as an active ingredient.

The senomorphics may restore the function of the senescent cells to normal cells.

The senescent cells may be selected from the group consisting of fibroblasts, umbilical vascular endothelial cells, retinal pigmented epithelial cells and renal proximal tubular cells, in which senescence is induced by drug treatment or subculture.

The present invention may comprise zotarolimus as an active ingredient, and the zotarolimus can provide a reagent composition for senomorphics which restores the function or morphology of senescent cells to normal cells in vitro.

In addition, the present invention can provide a method of restoring the function or morphology of senescent cells to normal cells, comprising treating zotarolimus on fibroblasts, umbilical vascular endothelial cells, retinal pigmented epithelial cells and renal proximal tubular cells isolated from mammals other than humans in vitro.

The present invention can provide a pharmaceutical composition for preventing or treating cellular senescence-associated diseases comprising zotarolimus as an active ingredient.

The zotarolimus may prevent or treat diseases induced by cellular aging by restoring a function or morphology of the senescent cells to normal cells.

The cellular senescence-associated disease may be selected from the group consisting of tissue fibrosis, senile eye disease, atherosclerosis, osteoarthritis, degenerative brain disease, obesity, diabetes and chronic skin damage.

The tissue fibrosis may be selected from the group consisting of renal fibrosis, pulmonary fibrosis and peritoneal fibrosis, but it is not limited thereto.

The senile eye disease may be selected from the group consisting of cataract, glaucoma and macular degeneration, but it is not limited thereto.

The degenerative brain disease may be selected from the group consisting of Parkinson's disease, Alzheimer's disease and stroke, but it is not limited thereto.

In one embodiment of the present invention, the pharmaceutical composition comprising zotarolimus as an active ingredient may be used as any one formulation selected from the group consisting injections, granules, powders, tablets, pills, capsules, suppositories, gels, suspensions, emulsions, drops or solutions according to the conventional method.

In another embodiment of the present invention, the pharmaceutical composition comprising zotarolimus as an active ingredient may further comprise at least one additive selected from the group consisting of carriers, excipients, disintegrants, sweeteners, coating agents, swelling agents, lubricants, slip modifiers, flavors, antioxidants, buffers, bacteristats, diluents, dispersants, surfactants, binders and lubricants, which are conventionally used for the preparation of the pharmaceutical composition.

Specifically, examples of the carrier, excipient and diluent include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oil. Solid formulations for oral administration include tablets, pills, powders, granules, capsules, etc., and such solid formulations may contain at least one excipient such as starch, calcium carbonate, sucrose or lactose, gelatin and the like in addition to the composition. Furthermore, in addition to simple excipients, lubricants such as magnesium stearate and talc are also used. Examples of the liquid formulations for oral administration include suspensions, solutions, emulsions, syrups and the like, and various excipients such as wetting agents, sweeteners, fragrances, preservatives and the like may be included in addition to water and liquid paraffin which are commonly used as simple diluents. Formulations for parenteral administration include sterile aqueous solutions, non-aqueous solutions, suspensions, emulsions, freeze-dried preparations, suppositories and the like. Examples of the non-aqueous solution and the suspension include propylene glycol, polyethylene glycol, vegetable oil such as olive oil, injectable ester such as ethyl oleate, and the like. As the base of the suppository, witepsol, macrogol, tween 61, cacao butter, laurinum, glycerogelatin and the like can be used.

According to one embodiment of the invention, the pharmaceutical composition may be administered intravenously, intraarterially, intraperitoneally, intramuscularly, intrasternally, transdermally, nasally, inhaled, topically, rectally, orally, intraocularlly or intradermally to the subject in the conventional manner.

The preferred dosage of zotarolimus may vary depending on the condition and weight of the subject, the type and extent of the disease, the drug form, the route of administration, and the duration, and may be appropriately selected by those skilled in the art. According to one embodiment of the present invention, the daily dosage may be, but is not limited to, 0.01 to 200 mg/kg, specifically 0.1 to 200 mg/kg, more specifically 0.1 to 100 mg/kg. Administration may be administered once a day or divided into several times, and the scope of the invention is not limited thereto.

In the present invention, the ‘subject’ may be a mammal including a human, but it is not limited thereto.

In addition, the present invention can provide a composition for improving aging or extending life, comprising zotarolimus as an active ingredient.

Hereinafter, the present invention will be described in more detail through examples. These examples are only intended to illustrate the present invention in more detail, and it will be apparent to those skilled in the art that the scope of the present invention is not limited by these examples according to the gist of the present invention. The examples of the present invention are provided to more completely explain the present invention to those of ordinary skill in the art.

The following experimental examples are intended to provide experimental examples commonly applied to each of the examples according to the present invention.

### <Experimental Example 1> Cell Culture

Human dermal fibroblast (HDF) and human umbilical vascular endothelial cells (HUVEC) were purchased from LONZA Inc. In (Walkersville, Md.), and human retinal pigmented epithelial cells (ARPE-19) and human tubular epithelial cell (HK2) were purchased from ATCC (Manassas, Va.) and used.

Human fibroblasts were cultured in DMEM (Dulbecco's Modified Eagle Medium) culture solution containing 10% fetal bovine serum (FBS), human umbilical vascular endothelial cells were cultured in EGM-2 culture solution, human retinal pigmented epithelial cells were cultured in DMEM:F12 culture solution containing 10% FBS, and HK-2 cells were cultured in RPMI1640 culture solution containing 10% FBS. Cells (2×10⁵) were dispensed into a 100 mm culture dish and subcultured in a 37° C., 5% CO₂ incubator.

When the cells grew to 80-90% in the culture dish, the cells were separated from the culture dish by treatment with a trypsin-EDTA solution, and the number of cells was measured. The degree of cell growth was confirmed by the cell population doubling time (PDT) as shown in the following equation.

PDT=((T−T₀)log 2)/(log N−log N₀)

(N=number of cells grown in the culture dish, N₀=number of first dispensed cells, T−T₀=cell culture time)

### <Experimental Example 2> Preparation of Premature Senescent Cells by Treatment with Doxorubicin

Each cell was treated in a serum-free culture solution containing 0.5 μM doxorubicin for 4 hours. After washing the cells with a serum-free culture solution, the cells were cultured for 4 days in a culture solution containing 10% FBS, and then cellular aging was confirmed by staining with senescence-associated beta galactosidase (SAβG).

### <Experimental Example 3> Preparation of Replicative Senescent Cells

After dispensing 2×10⁵ cells in a 100 mm culture dish, they were cultured in a 37° C., 5% CO₂ incubator. When the cells grew to 80-90% in the culture dish, the cells were removed by treatment with trypsin-EDTA, and the number of cells was measured, and the cell population doubling time (PDT) was measured.

By successively subculturing the cells in the same process as described above, replication aging was induced. PDT of young fibroblasts was 36 hours, PDT of replicative senescent fibroblasts was 12 days, PDT of young vascular endothelial cells was 24 hours, and PDT of replicative senescent vascular endothelial cells was 7 days.

The level of cellular senescence was confirmed by staining of senescence-associated beta galactosidase.

### <Experimental Example 4> Confirmation of Effective Substances of Senolytics and Senomorphics from Clinical Test Compounds

2,150 clinical trial compounds were distributed from Korea Chemical Bank.

Human fibroblasts and human vascular endothelial cells, in which premature senescence was induced by treatment with doxorubicin were dispensed into a 96-well plate, and then 2,150 compounds were treated at a concentration of 100 nM for 4 days, respectively.

The degree of survival of senescent cells was investigated by CCK-8 analysis, and the degree of senescence of cells was investigated by performing senescence-associated beta galactosidase staining.

### <Experimental Example 5> Drug Treatment

Zotarolimus was purchased from TOCRIS Bioscience (Minneapolis, Minn., USA), rapamycin was purchased from EMD Millipore (Burlington, Mass., USA).

After each sample was dissolved in DMSO, young cells, replicative senescent cells, and premature senescent cells treated with doxorubicin were treated with 100 nM zotarolimus, 100 nM rapamycin, 100 nM ABT263, respectively. After incubation for 4 days in a 37° C., 5% CO₂ incubator, the degree of cell growth was confirmed by CCK-8 analysis, the degree of cellular aging was confirmed by senescence-related beta-galactosidase (SAβG) staining,

### <Experimental Example 6> Analysis of Senescence-Associated β-Galactosidase (SAβG) Staining

The cells were washed with 1× phosphate buffer solution, and fixed with a phosphate buffer solution containing 3.7% (v/v) paraformaldehyde. After adding 1 mg/ml 5-bromo-4-chloro-3-indolyl-6-D-galactoside, 40 mM citric acid-sodium phosphate (pH 6.0), 5 mM potassium ferricyanide, 5 mM potassium ferrocyanide, 150 mM NaCl, and 2 mM MgCl₂ solution, it was reacted at 37° C. for 18 hours. It was stained with eosin as a control stain. Cells stained blue in the cytoplasm were identified using an optical microscope.

### <Experimental Example 7> Cell Survival Analysis (Cell Counting Kit-8 Assay; CCK-8)

Young cells (1×10³ cells/well) or senescent cells (2×10³ cells/well) were dispensed into a 96-well plate, and cultured overnight in a 37° C., 5% CO₂ incubator.

Zotarolimus, ABT263 and rapamycin were treated at each concentration, and then incubated for 4 days in a 37° C., 5% CO₂ incubator. 10 μl of CCK-8 reagent (Dojindo Molecular Technologies Inc., Kumamoto, Japan) was added to each well, and incubated for 2 hours in an incubator. Then, the absorbance was measured at 450 nm using a microplate reader.

The degree of cell growth was expressed as a relative value for the absorbance of the control group treated with only DMSO as 100%.

### <Experimental Example 8> Lactate Dehydrogenase Assay (LDH)

Cytotoxicity against young and senescent cells was investigated with a lactate dehydrogenase (LDH) activity kit (Dojindo Molecular Technologies Inc.).

Young cells (1×10³ cells/100 μl/well) or senescent cells (2×10³ cells/100 μl/well) were dispensed into a 96-well plate, and then cultured overnight in a 37° C., 5% CO₂ incubator. Zotarolimus and ABT263 were treated at each concentration, and then incubated for 4 days in a 37° C., 5% CO₂ incubator.

After transferring the cell culture solution to a 1.5 ml tube, it was centrifuged at 4° C. and 12,000 rpm. 100 μl of the supernatant was dispensed into a 96-well plate, and 100 μl of the LDH measurement solution was added to each well, followed by reaction in a CO₂ incubator for 30 minutes.

50 μl of the LDH reaction stop solution was added, and absorbance was measured at 490 nm using a microplate reader. The level of LDH activity was expressed as a relative value based on the absorbance of the control group treated with only DMSO.

### <Experimental Example 9> Preparation of Ischemia-Reperfusion Injury Induced Experimental Animal

Animal experiments were performed with the approval of the Animal Experimental Ethics Committee, Yeungnam University College of Medicine (YUMC-AEC2018-024). C57BL/6J 8-week-old male mice were anesthetized by intraperitoneal injection of 2.5% Avertin (0.025 ml/g body weight).

The anesthetized mice were placed on a hot plate at 37° C. and the left flank was incised to expose the left kidney. The renal arteriovenous blood vessels were ligated with a microaneurism clamp (Roboz), and whether the blood vessels were blocked or not was confirmed by the color of the kidneys. During the induction of ischemia, the body temperature of the mice was maintained at 36.5-37.5° C. After 35 minutes, the clamp was removed and it was confirmed that reperfusion occurred.

As a control, renal arteriovenous vessels were not ligated, and the rest was performed in the same manner as above. From 4 days after ischemia-reperfusion injury, 2.5 μl of 10 mM zotarolimus was diluted in 100 μl of phosphate buffer solution at intervals of 2 days and injected into the abdominal cavity 3 times (Zota group). The control group was a group that did not induce ischemia-reperfusion injury (Sham group) and an ischemia-reperfusion injury group (PBS) was used. Three mice were used for each group.

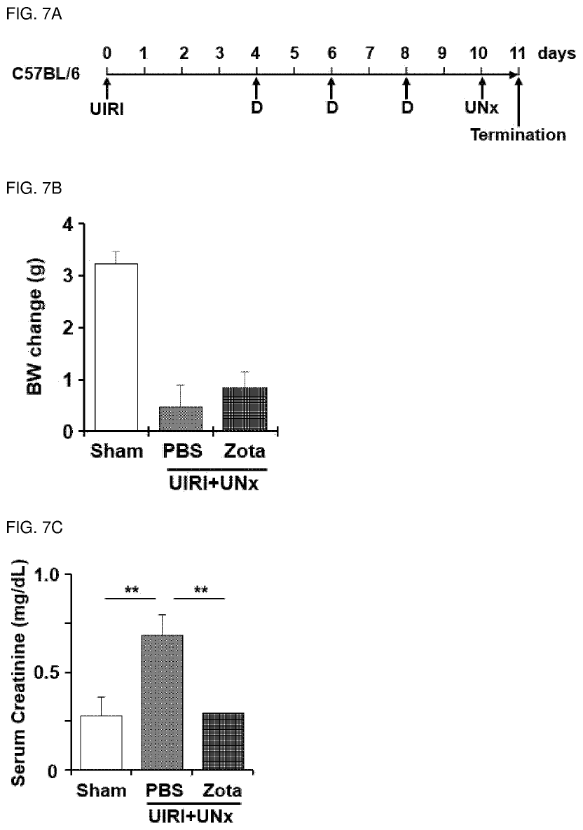

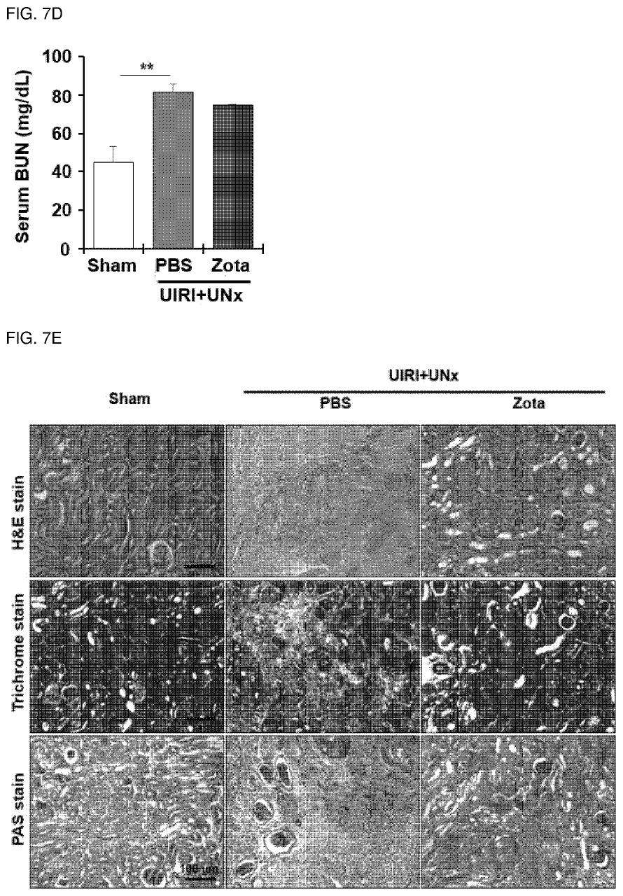

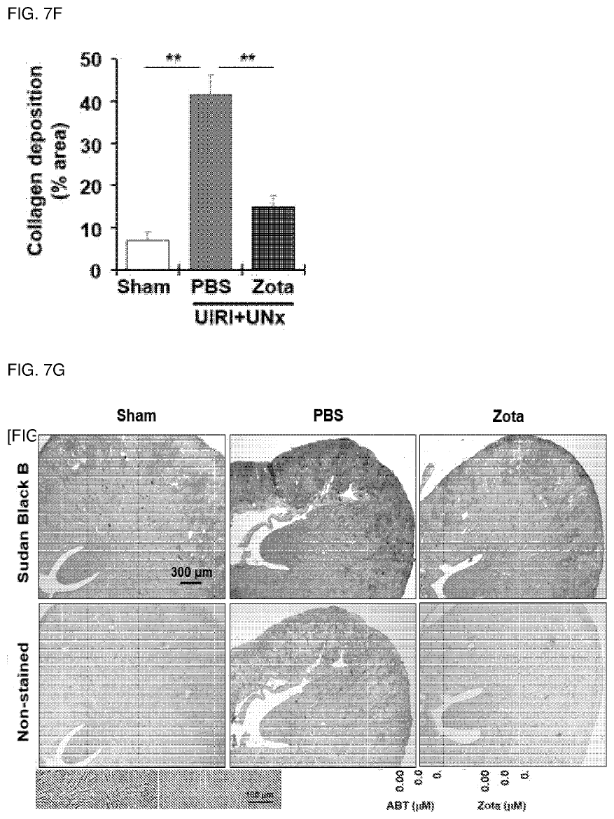

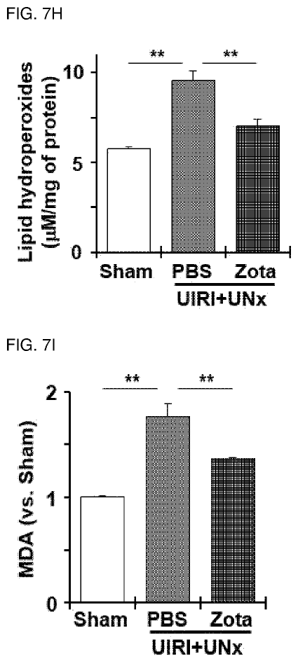

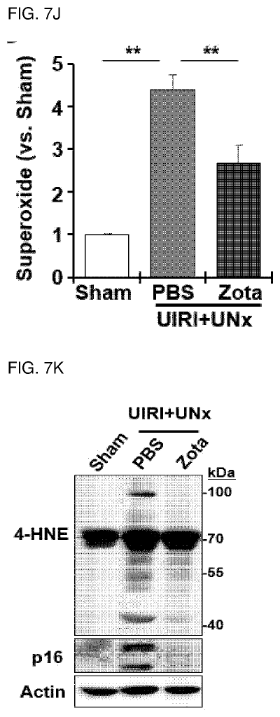

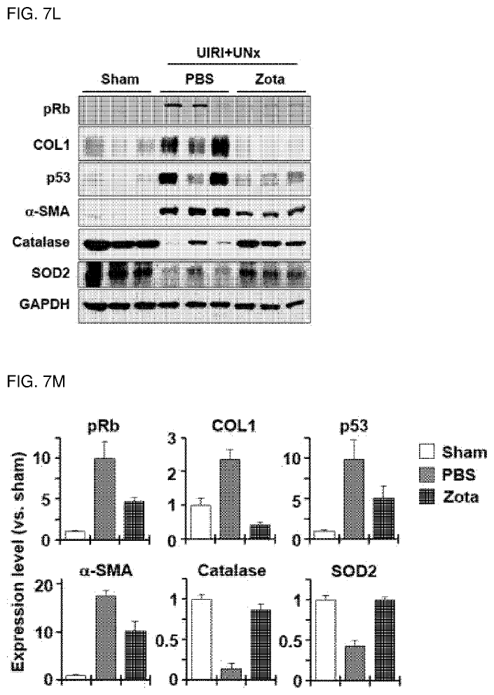

As shown in FIG. 7A, the right kidney was removed on the 10th day of the ischemia-reperfusion injury and sacrificed on the 11th day. The left kidney in which ischemia-reperfusion injury was induced, was excised in half and fixed in 10% formalin solution or stored frozen in liquid nitrogen.

### <Experimental Example 10> Evaluation of Kidney Function

In order to evaluate kidney function due to ischemia-reperfusion injury, creatinine and blood urea nitrogen (BUN) concentrations in plasma and urine were measured. Blood was collected from the retro-orbital vascular plexus of the mouse using a heparin capillary tube, and plasma was separated. Plasma creatinine was measured by QuantiChrom™ creatinine assay kit (DICT-500; BioAssay Systems, Hayward, Calif., USA), and BUN was investigated by measuring absorbance at 490 nm and 450 nm by a spectrophotometer, respectively by BUN colorimetric detection kit (Arbor Assays, Ann Arbor, Mich., USA).

### <Experimental Example 11> Confirmation of Malondialdehyde (MDA), Lipid Peroxides (Hydroperoxides) and Superoxides (O₂—) in Kidney Tissue

To confirm the degree of lipid peroxidation in tissues, malondialdehyde (MDA) was confirmed by the TBARS method (Garcia Y J, et al., Journal of neuroscience methods. 2005).

Briefly, 1.4 ml of TBARS solution [0.375% thiobarbituric acid (TBA), 15% trichloroacetic acid (TCA), 0.25 N HCl] was added to the tissue pulverization solution (0.3 mg protein/0.1 ml), and boiled for 15 minutes at 95-100° C. and centrifugation was performed at 4° C. and 12,000 rpm for 10 minutes, and the absorbance of the supernatant was confirmed at 540 nm.

Lipid hydroperoxides were identified by ferrous ion oxidation xylenol orange (FOX) method (Jiang Z Y, et al., Lipids. 1991).

0.9 ml of FOX reagent (100 μM xylenol orange, 25 mM H₂SO₄, 0.1 M sorbitol, 2.5 mM ferrous ammonium sulfate] was reacted at room temperature for 30 minutes and then centrifuged to measure the absorbance of the supernatant at 570 nm.

Tissue superoxide was measured using dihydroethidium (DHE; Sigma, St. Louis, Mo.) (Peshavariya H M, et al., Free radical research. 2007).

0.2 ml of 10 μM DHE was added to 0.2 ml of the tissue pulverization solution and reacted at room temperature for 10 minutes. Fluorescence was measured at 37° C. with an E max Precision Microplate Reader (Molecular Devices Corporation, Menlo Park, Calif., USA) at 544 nm for excitation and 612 nm for emission.

### <Experimental Example 12> Protein Extraction and Western Blot Analysis

RIPA buffer was added to the tissue and pulverized with WiseTis homogenizer HG-15D (DAIHAN Scientific, Seoul, South Korea). Centrifugation was performed at 12,000 rpm at 4° C. for 20 minutes, and the supernatant was transferred to a new tube.

The protein concentration of the supernatant was quantified by the BCA method. After electrophoresis (SDS-PAGE) of 30 μg of protein, Western blot analysis was performed. After electrophoresis, the protein was transferred from the gel to a polyvinylidene fluoride membrane (Pall Corporation).

The membrane was treated with a 5% Difco™ skim milk solution (Becton, Dickinson and Company, USA) for 2 hours at room temperature, and the primary antibody was added, followed by reaction at room temperature for 2 hours. As the primary antibody, anti-p53 antibody, anti-p16 antibody, anti-actin antibody, and anti-GAPDH antibody were purchased from Santa Cruz Biotechnology, Inc. Anti-phosphorylated-Rb antibody was obtained from New England Biolabs (Ipswich, Mass., USA), and the anti-SOD2 antibody and anti-catalase antibody were obtained from Bioworld Technology Inc. (Louis Park, Minn., USA), anti-4-HNE antibody, anti-α-smooth muscle actin antibody, and anti-type I collagen antibody were purchased from Abcam (Cambridge, UK).

The membrane treated with the primary antibody was washed three times for 15 minutes each with TBST (20 mM Tris-HCl (pH 8.0), 137 mM NaCl, 0.1% Tween 20), and reacted for 60 minutes by treatment with the secondary antibody.

After washing for at least 60 minutes with TBST, protein expression was confirmed using an ECL detection kit (Elpis Biotech, Daejeon, South Korea).

### <Experimental Example 13> Preparation of Experimental Animals for Lung Fibrosis

Animal experiments were conducted with the approval of the Animal Experimental Ethics Committee, Yeungnam University College of Medicine (YUMC-AEC2018-025). After anesthetizing by injecting 2.5% avertin (Avertin; 0.025 ml/g) into the abdominal cavity of 10-week-old C57BL/6J male mice, 2 mU/g of bleomycin was instilled into the bronchi and injected into the lungs.

After bleomycin instillation, the weight was measured on the 3rd, 5th and 7th days, and mice that did not lose weight were excluded from the experiment. From 5 days after bleomycin administration, 2.5 μl of 10 mM zotarolimus was diluted in 100 μl phosphate buffer solution at intervals of 3 days and injected intraperitoneally 6 times (Zota group).

On the other hand, the group that did not induce pulmonary fibrosis (NT group) and the group that injected only 2.5 μl DMSO-containing phosphate buffer (PBS) after bleomycin administration (PBS group) were used as a control group.

Three mice were used for each group, and lungs were excised by sacrifice 22 days after bleomycin injection and fixed in 10% formalin solution or stored frozen in liquid nitrogen.

### <Experimental Example 14> Preparation of Experimental Animal for Peritoneal Fibrosis

Animal experiments were conducted with the approval of the Animal Experimental Ethics Committee of Yeungnam University College of Medicine (YUMC-AEC2018-033). Peritoneal fibrosis mice experiments were performed using a model of peritoneal fibrosis induced by chlorhexidine gluconate (CHG) (Yoshio Y et al., Kidney international. 2004).

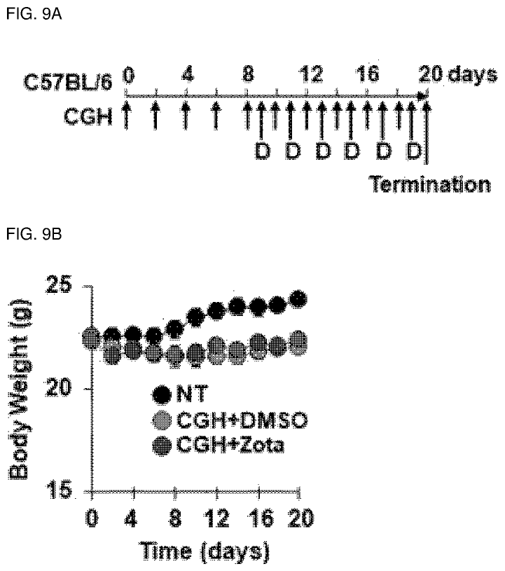

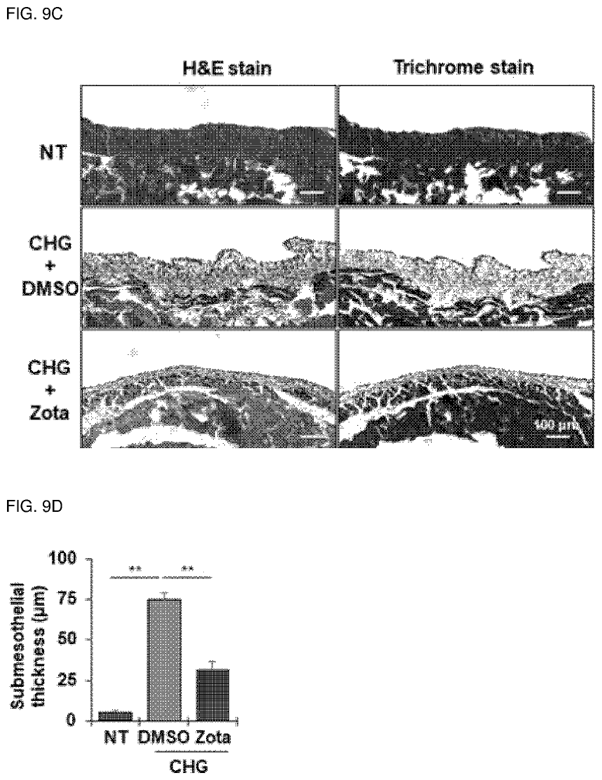

As shown in FIG. 9A, C57BL/6J 8-week-old male mice were intraperitoneally injected with a 0.1% CHG solution (0.1% CHG in a 15% ethanol phosphate buffer solution) at 10 ml/kg for 20 days at intervals of 2 days. From the 9th day, 2.5 μl of 10 mM zotarolimus (Zota group) or 2.5 μl of DMSO (DMSO group) was diluted in 100 μl phosphate buffer solution and intraperitoneally injected at intervals of 2 days.

As a negative control (NT group), 100 μl of a 15% ethanol phosphate buffer solution was injected intraperitoneally instead of a 0.1% CHG solution. On the 20th day, the mice were sacrificed, the peritoneal tissue was excised, and the tissue was fixed in 4% paraformaldehyde solution.

### <Experimental Example 15> Preparation and Staining of Tissue Samples

The tissue fixed in 10% formalin solution was embedded with paraffin and cut into 4 μm to prepare a tissue sample. After removing paraffin from the tissue sample, hematoxylin-eosin staining, trichrome staining, and PAS staining were performed.

The degree of fibrosis was measured by analyzing the degree of collagen staining using the i-Solution™ software program (IMT Inc., Canada) after trichrome staining and the degree of cell senescence in tissue samples was investigated by staining lipofuscin with Sudan black B (Viegas M S, et al., European journal of histochemistry: EJH. 2007). The degree of peritoneal fibrosis was confirmed by measuring the submesothelial thickness of the peritoneal mesothelial cell layer using the NIH Image J program.

### <Example 1> Confirmation of Senomorphics Action of Zotarolimus

1. Confirmation of Senomorphics Action of Zotarolimus on Human Fibroblasts

Human fibroblasts were treated with doxorubicin to induce early cellular senescence. Cells in which early senescence was induced were treated with 0.01% DMSO (NT), 100 nM zotarolimus (Zota), 100 nM rapamycin (Rapa) and 100 nM ABT263 (ABT), and 4 days later, SAβG activity staining was performed to confirm the degree of senescence of the cells, and the degree of cell growth was confirmed by performing CCK-8 analysis.

Rapamycin is a type of senomorphics known to inhibit cellular aging, and ABT263 is a type of senolytics known to induce senescent cell-specific apoptosis, and the above two drugs were used as positive controls.

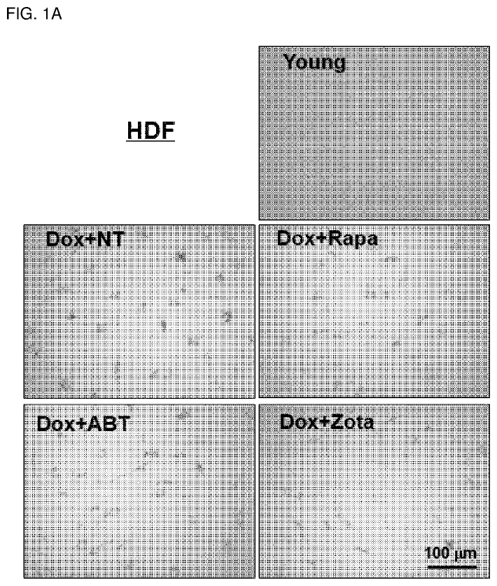

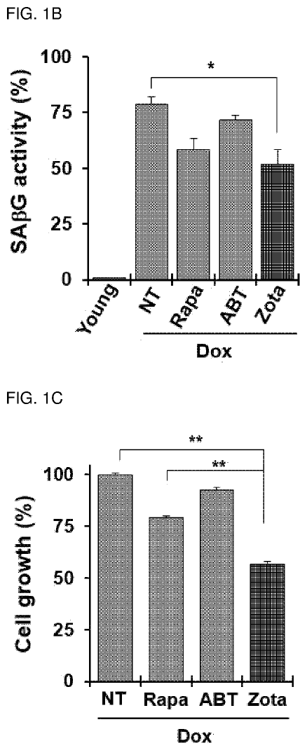

As a result, when 100 nM of zotarolimus was treated in human fibroblasts in which early cell senescence was induced by doxorubicin treatment as shown in FIG. 1A and FIG. 1B, SAβG activity was decreased compared to the DMSO-treated group, and as shown in FIG. 1C, cell growth was also significantly decreased (*, p<0.05, **, p<0.01).

In addition, it was confirmed that zotarolimus reduces senescent cell-specific SABG activity and cell growth in premature senescent human fibroblasts as well as replicative senescent human fibroblasts.

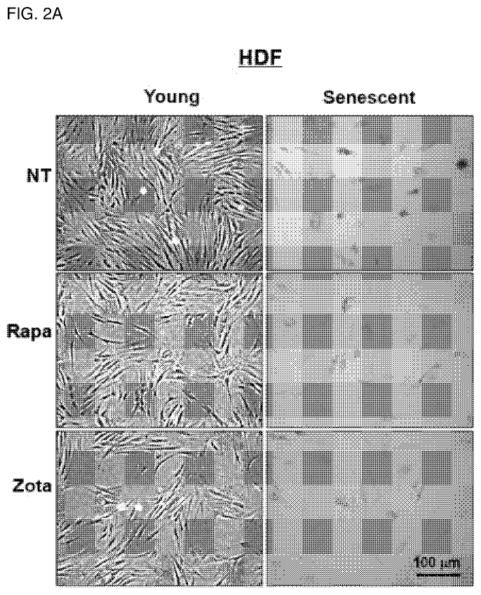

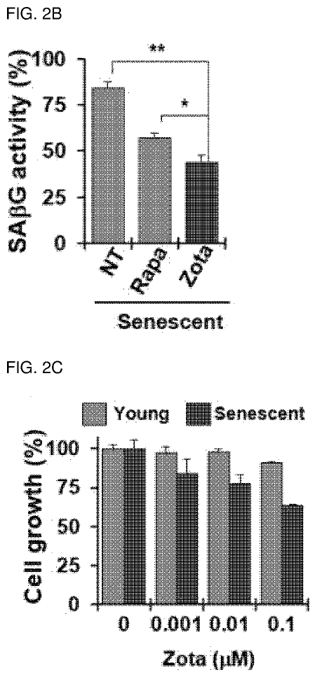

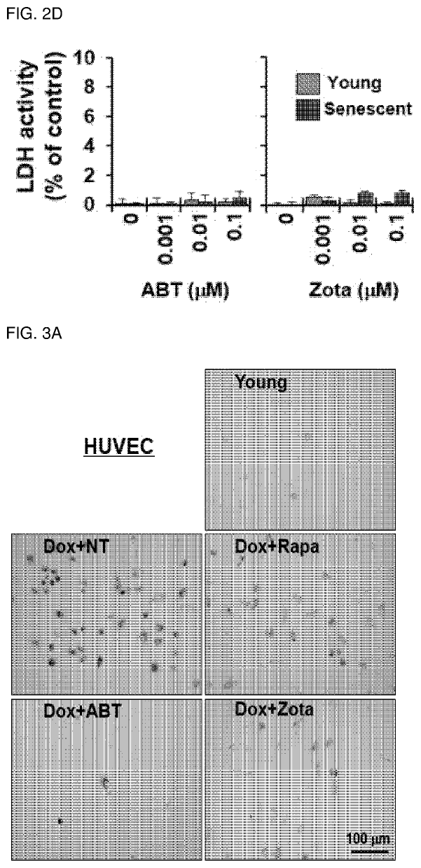

As a result of confirming the cells treated with the drug in the same manner as the cells in which replication senescence was induced, SAβG activity was significantly reduced (**, p<0.01) by treatment with zotarolimus in replicative senescent fibroblasts as shown in FIG. 2A and FIG. 2B and it could be confirmed that it was significantly reduced than that of rapamycin (*, p<0.05).

In addition, when young fibroblasts and replicative senescent fibroblasts were treated with increasing the concentration of zotarolimus, it was confirmed that cell growth was more markedly decreased in senescent cells compared to young cells in a concentration-dependent manner as shown in FIG. 2A and FIG. 2C.

From the above results, in order to confirm whether the decrease in cell growth caused by zotarolimus was due to cell death or simply because the growth stopped, LDH activity was investigated in the cell culture solution.

As a result, as it was confirmed that there was no difference in LDH activity in the cell culture medium as shown in FIG. 2D, instead of selectively killing senescent cells in human fibroblasts, zotarolimus can restore the function and morphology of senescent cells to young cells and thus it has been identified as a drug that acts as a new senomorphics that can restore the function and morphology of senescent cells to young cells

2. Confirmation of Senomorphics Action of Zotarolimus on Human Umbilical Vascular Endothelial Cells

Human umbilical vascular endothelial cells were treated with doxorubicin to induce early cellular aging, and then early aged cells were treated with 0.01% DMSO, 100 nM zotarolimus, 100 nM rapamycin and 100 nM ABT263, and after 4 days, SAβG activity staining was performed to confirm the degree of cellular aging, and the cell growth was confirmed by CCK-8 analysis.

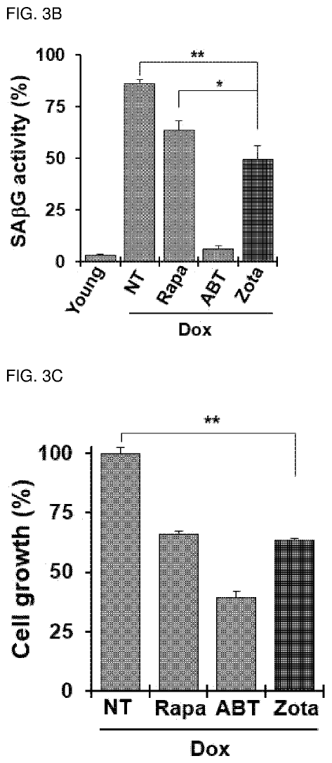

As a result, as shown in FIG. 3A and FIG. 3B, it was confirmed that the SAβG activity stating of the experimental group in which human vascular endothelial cells in which early cellular aging was induced by doxorubicin treatment was treated with 100 nM zotarolimus was significantly decreased (**, p<0.01) compared to that of the DMSO-treated group and zotarolimus was significantly decreased than rapamycin.

In addition, as shown in FIG. 3C, zotarolimus significantly reduced the cell growth of premature senescent vascular endothelial cells than that of DMSO (**, p<0.01), but it was confirmed that it exhibited a similar effect to rapamycin.

Based on the above results, it was confirmed whether zotarolimus acts as a senomorphics for not only early senescent cells but also replicative senescent vascular endothelial cells.

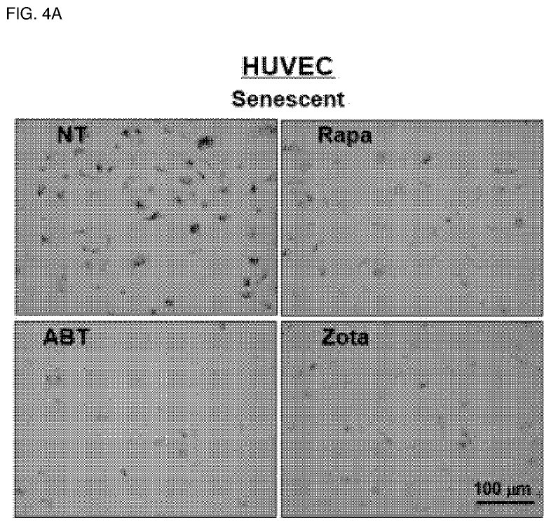

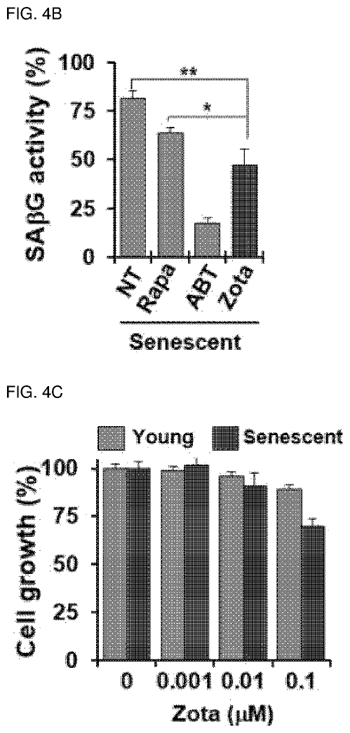

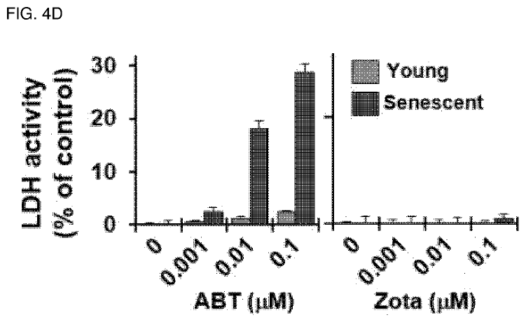

As a result, as shown in FIG. 4A and FIG. 4B, SAβG activity in replicative senescent vascular endothelial cells by zotarolimus treatment was significantly decreased (*, p<0.05; **, p<0.01) compared to that of the DMSO-treated group and the rapamycin-treated group. In addition, when the young vascular endothelial cells and the replicative senescent vascular endothelial cells were treated with increasing the concentration of zotarolimus, the cell growth of senescent cells was decreased compared to the young cells in a concentration-dependent manner as shown in FIG. 4C.

In order to confirm whether the decrease in cell growth caused by zotarolimus as described above was due to cell death, LDH activity was investigated in the cell culture solution.

As a result, there was no difference in LDH activity in the cell culture solution as shown in FIG. 4D.

From the above results, it was confirmed that, instead of selectively killing senescent cells in human vascular endothelial cells, zotarolimus acts as a senomorphics capable of restoring the function and morphology of senescent cells to young cells.

3. Confirmation of Senomorphics Action of Zotarolimus on Human Retinal Pigmented Epithelial Cells

Human retinal pigmented epithelial cells were treated with doxorubicin to induce early cellular aging, and then early aged cells were treated with 0.01% DMSO, 100 nM zotarolimus and 100 nM rapamycin and 4 days later, SAβG activity staining was performed to confirm the cellular aging level and the degree of cell growth was confirmed by CCK-8 analysis.

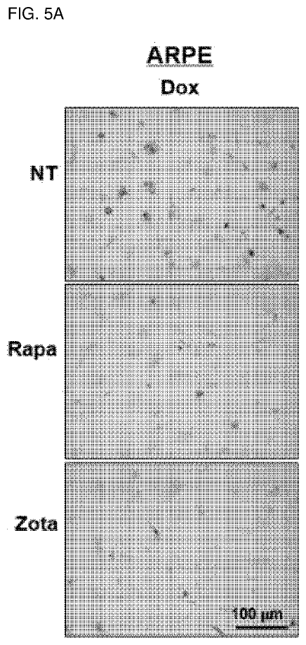

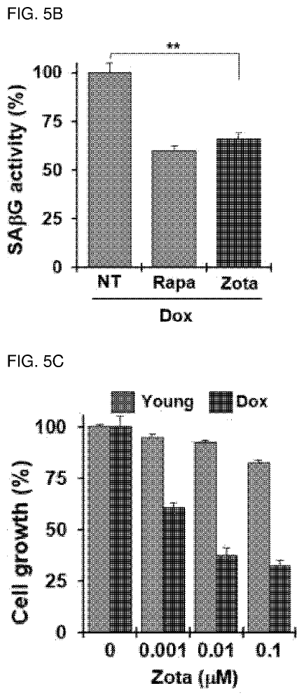

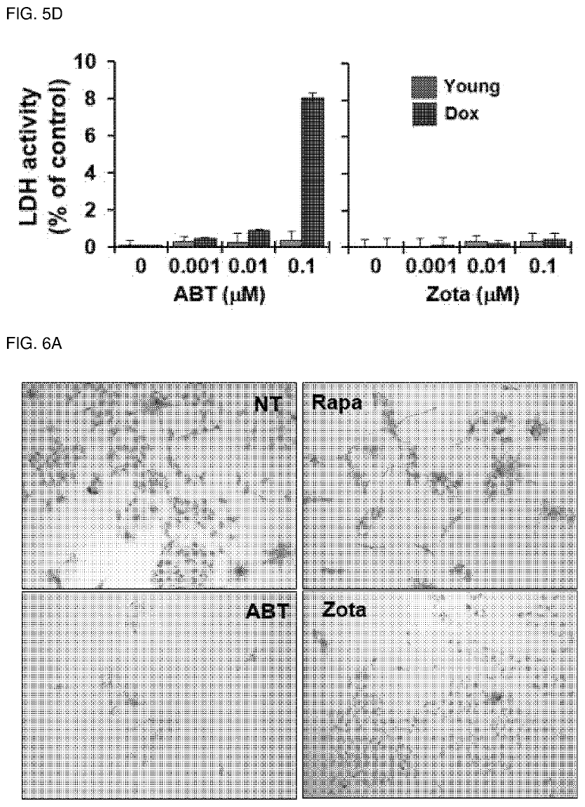

As a result, as shown in FIG. 5A and FIG. 5B, the SAβG activity staining of the experimental group in which human retinal pigmented epithelial cells induced with early cellular aging was treated with zotarolimus significantly decreased (**, p<0.01) compared to that of the DMSO-treated group

In addition, when the young retinal pigment epithelial cells and the replicative senescent retinal pigmented epithelial cells were treated with increasing concentrations of zotarolimus, as shown in FIG. 5C, like fibroblasts and vascular endothelial cells, the cell growth was significantly decreased in senescent cells compared to young cells in a concentration-dependent manner.

On the other hand, as a result of examining whether the cell growth inhibitory effect of zotarolimus in senescent cells is due to cytotoxicity by LDH activity analysis, it was confirmed that cytotoxicity specific to senescent cells did not appear as shown in FIG. 5D.

From the above results, it was confirmed that, instead of selectively killing senescent cells in human retinal pigmented epithelial cells, zotarolimus acts as a senomorphics capable of restoring the function and morphology of senescent cells to young cells.

4. Confirmation of Senomorphics Action of Zotarolimus in Human Proximal Renal Tubular Cells

After treating HK2 cells with doxorubicin to induce early cell senescence, premature senescent cells were treated with 0.01% DMSO, 100 nM zotarolimus, 100 nM rapamycin and 100 nM ABT263, and 4 days later, the level of senescence of cells was increased by SAβG activity staining and the cell growth was confirmed by CCK-8 analysis.

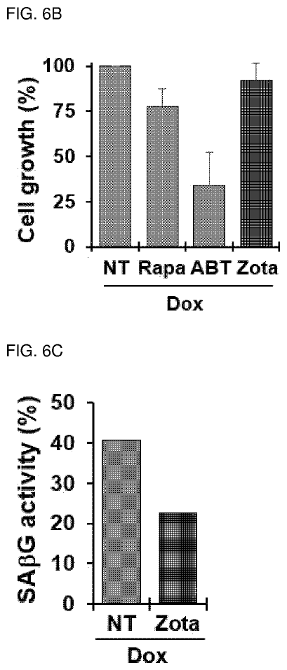

As a result, as shown in FIG. 6A and FIG. 6B, it was confirmed that in the cell group treated with zotarolimus, there was no difference in the growth of premature senescent HK2 cells compared to the DMSO-treated group (**, p<0.01), but SAβG staining decreased as shown in FIG. 6C.

From the above results, it was confirmed that in human HK2 cells, zotarolimus acts as a senomorphics capable of restoring the function and morphology of senescent cells to young cells, instead of selectively killing senescent cells.

### <Example 2> Confirmation of the Effect on Tissue Fibrosis According to Cell Aging

1. Confirmation of Efficacy of Zotarolimus on Renal Fibrosis Induced by Renal Ischemia-Reperfusion Injury

As renal fibrosis induced by renal ischemia-reperfusion injury is reported to be related to the cellular aging of the tissue. The left renal blood vessels of mice were ligated and released for 35 minutes to induce ischemia-reperfusion injury.

After 4 days, 2.5 μl of 10 mM zotarolimus was diluted in 100 μl phosphate buffer solution at intervals of 2 days and injected intraperitoneally 3 times (Zota group). A group that did not induce ischemia-reperfusion injury (Sham group) and an ischemia-reperfusion injury group (PBS) were designated as a control group, and as shown in FIG. 7A, the right kidney was removed on the 10th day of the ischemia-reperfusion injury, and sacrificed on the 11th day, and the body weight before the ischemia-reperfusion injury and the body weight change on the 11th day were investigated.

As a result, as shown in FIG. 7B, the weight gain of the PBS group or the Zota group was significantly reduced compared to that of the Sham group. In addition, as shown in FIG. 7C and FIG. 7D, it was confirmed that plasma creatinine and BUN were decreased in the Zota group compared to those in the PBS group.

On the other hand, as a result of performing a kidney tissue specimen examination, damage to the kidney tubules was significantly reduced in the Zota group compared to that in the PBS group, as shown in FIG. 7E and FIG. 7F, and as a result of confirming the degree of tissue fibrosis by trichrome staining, it was also significantly reduced in the Zota group compared to the PBS group.

As lipofuscin is known to increase when cell aging occurs in tissues and it has been reported that cell aging can be measured enough to replace SAβG staining, lipofuscin was stained with Sudan Black B in tissues.

As a result, it was found that the staining intensity was significantly reduced in the Zota group compared to that in the PBS group, as shown in FIG. 7G.

In addition, the tissue was crushed and the degree of oxidation of lipids was confirmed by lipid hydroperoxides and MDA.

As a result, as shown in FIG. 7H and FIG. 7I, the degree of lipid oxidation was significantly reduced in the Zota group compared to that in the PBS group, and as shown in FIG. 7J, superoxide, a kind of active oxygen, was also significantly reduced in the Zota group.

On the other hand, the protein was isolated from the tissue, and the expression level of p16 as a cell aging label and 4-HNE as an oxidative stress label was confirmed by Western blot analysis.

As a result, it was found that the expression levels of p16 and 4-HNE in the Zota group were significantly reduced compared to those of the PBS group, as shown in FIG. 7K, and referring to FIG. 7L, the expressions of pRb as a cell proliferation marker, type 1 collagen as a fibrosis marker (COL1), α-SMA, and p53 as a cell aging marker were also confirmed to be significantly reduced in the Zota group compared to those in the PBS group.

In addition, as shown in FIG. 7M, it was confirmed that the expressions of antioxidant enzymes catalase and SOD2 were increased in the Zota group.

From the above results, it was confirmed that zotarolimus restores the function and morphology of senescent cells observed in kidney ischemia-reperfusion injury, thereby exhibiting an effect of inhibiting renal fibrosis.

2. Analysis of Efficacy of Zotarolimus on Pulmonary Fibrosis by Bleomycin

It is well known that bleomycin-induced lung fibrosis in mice is associated with cell aging in lung tissue.

First, the body weight before induction of pulmonary fibrosis and the body weight change on the 20th day were confirmed.

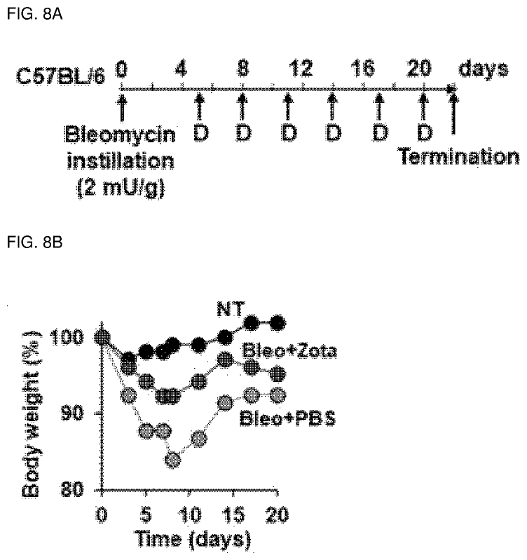

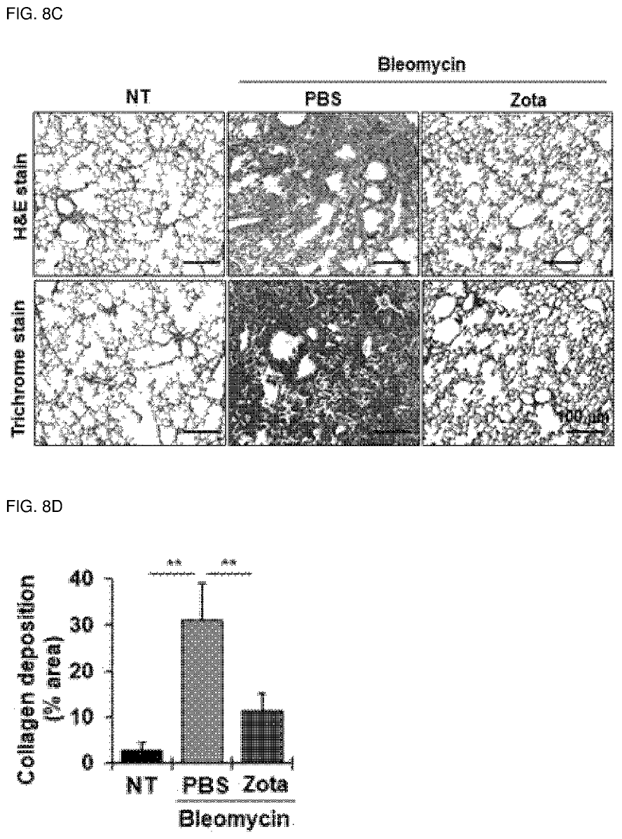

As a result, it was found that the weight gain was significantly decreased in the PBS group and the Zota group compared to that in the NT group as shown in FIG. 8A, but referring to FIG. 8B, it was confirmed that the weight of the Zota group was further increased compared to that of the PBS group.

In addition, lung tissue specimens were prepared, and hematoxylin-eosin staining and trichrome staining were performed to confirm the degree of damage to the lung tissue and the degree of lung fibrosis.

As a result, it was confirmed that the degree of lung tissue damage and lung fibrosis were statistically significantly reduced in the Zota group compared to those in the PBS group as shown in FIG. 8C and FIG. 8D.

From the above results, it was confirmed that zotarolimus exhibits an effect of inhibiting pulmonary fibrosis induced by bleomycin.

3. Confirmation of Efficacy of Zotarolimus on Peritoneal Fibrosis Induced by Chlorhexidine Gluconate (CHG)

Peritoneal fibrosis is a side effect that occurs frequently in patients who have undergone peritoneal dialysis, and may cause a problem of reducing peritoneal dialysis efficiency.

Long-term peritoneal dialysis increases reactive oxygen species by components of peritoneal dialysis fluid, and induces peritoneal fibrosis due to chronic inflammation. In this process, epithelial to mesenchymal transition of peritoneal mesenchymal cells by TGF-β1 is known to play an important role, and TGF-β1 is well known to induce cellular aging.

In order to confirm the effect of zotarolimus on the peritoneal fibrosis, a 0.1% CHG solution was injected intraperitoneally for 20 days at intervals of 2 days, and zotarolimus or DMSO-phosphate buffer solution was injected intraperitoneally from the 9th day and abdominal wall tissue specimens were prepared, and hematoxylin-eosin staining and trichrome staining were performed, and then the thickness of the peritoneal mesothelial cell layer and the degree of peritoneal fibrosis were analyzed.

As a result, it was confirmed that the thickness of the peritoneal mesothelial cell layer in the zotarolimus-treated group (Zota group) was significantly reduced compared to that in the DMSO-phosphate buffer solution (DMSO group) as shown in FIG. 9C and FIG. 9D, and the degree of fibrosis was also decreased.

From the above results, it was confirmed that zotarolimus inhibits peritoneal fibrosis induced by CHG.

While the present invention has been particularly described with reference to specific examples thereof, it is apparent that this specific description is only a preferred example and that the scope of the present invention is not limited thereby to those skilled in the art. Accordingly, the practical scope of the present invention is defined by the appended claims and their equivalents.

1. A composition having a senomorphic activity comprising zotarolimus as an active ingredient.

2. The composition of claim 1, wherein the senomorphic activity restores function of a senescent cells to a normal cells.

3. The composition of claim 2, wherein the senescent cell is selected from the group consisting of a fibroblast, an umbilical vascular endothelial cell, a retinal pigmented epithelial cell, and a renal proximal tubular cell, and wherein a senescence is induced by drug treatment or subculture in the senescent cell.

4. A method for preventing or treating a cellular senescence-associated disease comprising administering a pharmaceutical composition comprising zotarolimus as an active ingredient to a subject in need of the preventing or treating the cellular senescence-associated disease.

5. The method of claim 4, wherein the zotarolimus selectively kills a senescent cell or restores function or morphology of the senescent cell to a normal cells to prevent or treat the cellular senescence-associated disease.

6. The method of claim 4, wherein the cellular senescence-associated diseases is selected from the group consisting of a tissue fibrosis, a senile eye disease, an atherosclerosis, an osteoarthritis, a degenerative brain disease, an obesity, a diabetes and a chronic skin damage.

7. The method of claim 6, wherein the tissue fibrosis is selected from the group consisting of a renal fibrosis, a pulmonary fibrosis, and a peritoneal fibrosis.

8. A method for improving aging or extending life comprising administering a pharmaceutical composition comprising zotarolimus as an active ingredient to a subject in need of the improving aging or extending life.
